(19) United States
(12) Patent Application Publication
(10) Pub. No.: US 2003/0118571 A1
(43) Pub. Date: Jun. 26, 2003

(54) Oral administration of lactobacillus for the maintenance of health in women during pregnancy and at other life stages, to reduce the risk of urogenital diseases

(76) Inventors: Gregor Reid, London (CA); Andrew W. Bruce, Uxbridge (CA)

Correspondence Address: SCULLY SCOTT MURPHY & PRESSER, PC, 400 GARDEN CITY PLAZA, GARDEN CITY, NY, 11530

(21) Appl. No.: 10/317,666

(22) Filed: Dec. 10, 2002

Related U.S. Application Data
(62) Division of application No. 09/579,307, filed on May 25, 2000, now abandoned.
(60) Provisional application No. 60/135,955, filed on May 25, 1999.

(51) Int. Cl. A61K 31/665; A01N 63/00

(52) U.S. Cl. 424/93.45; 424/93.4; 514/100

(57) ABSTRACT

The present invention provides methods and compositions for the oral administration of Lactobacillus and/or other probiotic organisms, such as Bifidobacterium, for establishment and maintenance of a healthy urogenital flora. The invention also provides methods and compositions to reduce the risk of disease, including onset of preterm labor due to vaginal and cervical infection. The invention also provides ex vivo methods of restoring healthy gastrointestinal and vaginal flora.

### CROSS REFERENCE TO RELATED APPLICATION

This application claims the benefit of U.S. Provisional Application No. 60/135,955 filed May 25, 1999.

### FIELD OF THE INVENTION

The present invention provides methods and compositions for the oral administration of Lactobacillus and/or other probiotic organisms, such as Bifidobacterium, for establishment and maintenance of a healthy urogenital flora. The invention also provides methods and compositions to reduce the risk of disease, including onset of preterm labor due to vaginal and cervical infection.

### BACKGROUND OF THE INVENTION

The organisms which constitute the flora of the urogenital tract in females, originate from the gastrointestinal tract. Of the approximately 50 species of microbes which inhabit the vagina, urethra, cervix, perineum and vulva, Lactobacillus and Bifidobacterium represent the most dominant species in healthy women. These organisms dominate the vaginal flora in pre-menopausal women. The ability of these organisms to exist, persist and dominate the flora, is influenced in part by the changing bacterial and nutrient environment of the gastrointestinal tract.

In post-menopausal women, the normal healthy urogenital flora may also contain lactobacilli, bifidobacteria, or other naturally occurring and non-infectious organisms. However, in this age group, urogenital infections are particularly common, and some studies have suggested that the reduction in estrogen levels causes the depletion of lactobacilli, due in part to reduced amounts of glycogen or mucus which lactobacilli use as nutrients and receptors.

Organisms, e.g. urogenital pathogens, which cause many urogenital infections, such as yeast vaginitis, bacterial vaginosis and urinary tract infections, originate predominantly in the gastrointestinal tract. In such disease states, the lactobacilli flora are depleted. Urogenital infections predispose many pregnant women to preterm labor and premature birth. In young and aged females alike, a disruption of the urogenital flora leads to an increased risk of sexually transmitted diseases. Thus, establishment and maintenance of a normal, healthy urogenital flora is vitally important to the well-being of females.

Previous studies have shown that certain lactobacilli organisms possess the ability to interfere with urogenital pathogens. (Reid, et al. (1987) “Examination of strains of lactobacilli for properties which may influence bacterial interference in the urinary tract,” J. Urol., 138:330-335; Reid, et al. (1988) “Lactobacillus inhibitor production against E. coli and coaggregation ability with uropathogens,” Can. J. Microbiol., 34:344-351). Such lactobacilli can be delivered orally and vaginally to prevent infections. The delivery of bacteria to improve well being is termed probiotics.

### SUMMARY OF THE INVENTION

The present invention provides methods and compositions for the establishment and maintenance of a healthy gastrointestinal and urogenital flora. The invention provides lactobacilli or bifidobacteria, which, when taken orally, enhance the flora's ability to out-compete gastrointestinal and urogenital pathogens. From this intestinal niche, the probiotic organisms unexpectedly emerge to naturally colonize the perineum, vulva, vagina and/or urethra and to establish and maintain a normal healthy flora.

In one aspect of the present invention a method is provided for establishing a healthy gastrointestinal and urogenital flora in females throughout life comprising orally administering a therapeutically effective amount of at least one probiotic organism and a pharmaceutically acceptable carrier. In a further aspect of the method a therapeutically effective amount of a second probiotic organism is administered. Lactobacillus is the preferred probiotic organism. The Lactobacillus is preferably selected from the group consisting of L. rhamnosus, L. acidophilus, L. fermentum, L. casei, L reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosae, or L. bifidum. Bifidobacteria is the preferred second probiotic organism. The Bifidobacterium is preferably selected from the group consisting of B. bifidum, B. breve, B. adolescentis, or B. longum.

In another aspect of the present invention a prebiotic is administered in conjunction with the probiotic organism.

In still another aspect of the present invention an ex vivo method is provided for establishing a healthy gastrointestinal and urogenital flora in a females comprising orally administering at least one probiotic organism isolated from said female and a pharmaceutically acceptable carrier. In a further aspect the probiotic organisms are isolated or obtained from the patient.

In yet another aspect of the present invention a method is provided for maintaining a healthy urogenital flora in females prior to, during and after pregnancy comprising orally administering at least one probiotic organism and a pharmaceutically acceptable carrier. In a further aspect of the method a therapeutically effective amount of a second probiotic organism is administered. Lactobacillus is the preferred first probiotic organism. The Lactobacillus is preferably selected from the group consisting of L. rhamnosus, L. acidophilus, L. fermentum, L. casei, L reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosae, or L. bifidum. Bifidobacteria is the preferred second probiotic organism. The Bifidobacterium is preferably selected from the group consisting of B. bifidum, B. breve, B. adolescentis, or B. longum.

In still another aspect of the present invention an ex vivo method is provided for restoring healthy gastrointestinal and urogenital flora in females in need thereof comprising orally administering at least one probiotic organism isolated from the individual and a pharmaceutically acceptable carrier.

In another aspect of the present invention, a method is provided for reducing the risk of preterm labor comprising orally administering a therapeutically effective amount of at least one probiotic organism and a pharmaceutically acceptable carrier.

In another aspect of the present invention, a method is provided for reducing the risk of bacterial vaginosis and bacterial vaginosis pathogens comprising orally administering a therapeutically effective amount of at least one probiotic organism and a pharmaceutically acceptable carrier.

In still yet another aspect of the present invention a pharmaceutical composition is provided which comprises a probiotic organism and a pharmaceutically acceptable carrier.

In yet another aspect of the present invention an ex vivo method is provided for maintaining healthy urogenital flora in a newborn comprising orally administering at least one probiotic organism which has been isolated from the newborn or mother or provided from an exogenous source and a pharmaceutically acceptable carrier.

In a further aspect of the present invention a method is provided for selecting lactobacilli and bifidobacteria useful for improving gastrointestinal and urogenital health comprising detecting an ability to: adhere to gastrointestinal, vaginal and uroepithelial cells by electrostatic, hydrophobic or specific adhesins including a collagen binding protein; pass through the stomach and reach the small and large intestine; grow and persist in the gastrointestinal and urogenital tracts; inhibit the adhesion of gastrointestinal an urogenital pathogens including organisms which cause urinary tract infection, bacterial vaginosis and yeast vaginitis; coaggregate to form a balanced flora; produce acid and other substances such as hydrogen peroxide and/or bacteriocins and bacteriocin-like compounds which inhibit pathogen growth; produce biosurfactant or related by-products of growth which interfere with adhesion of pathogens to cells and materials; resist antimicrobial agents, such as nonoxynol-9 spermicide; and/or enhance the host's immune function to further maintain a healthy urogenital flora.

### BRIEF DESCRIPTION OF THE FIGURES

FIG. 1 is a bar graph which shows viable counts of lactobacilli strains before (white bars) and after (dark bars) 16-hour incubation with FALL-39 (100 umol 1⁻¹). Mottled bars represent control counts (incubation without peptide). Mean values obtained from three independent experiments are shown.

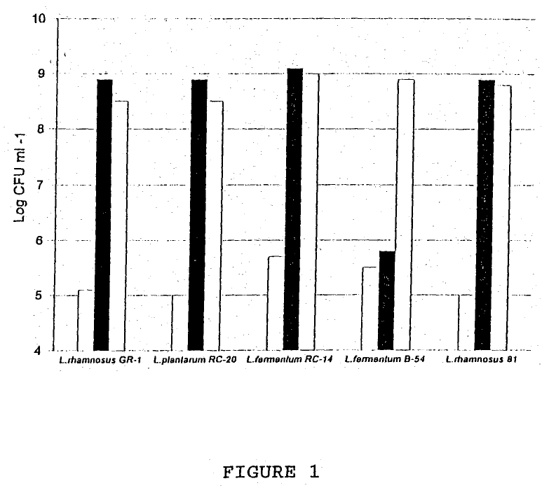

FIG. 2 is a surface enhanced laser desorption/ionization (SELDI) mass profile of lactobacillus expression of collagen binding proteins in Lactobacillus acidophilus RC-14; L. rhamnosus GR-1 and L. rhamnosus 36W.

FIG. 3 is a SELDI mass profile of lactobacillus expression of collagen binding proteins in Lactobacillus acidophilus RC-14 using protein chip PS-1/CN-III.

### DETAILED DESCRIPTION OF THE INVENTION

The present invention provides methods and compositions for establishing and maintaining a healthy gastrointestinal and urogenital flora in women throughout the life cycle comprising the administration of probiotic organisms such as Lactobacillus and/or Bifidobacterium and/or a prebiotic compound.

By “probiotic” is meant an organism which has one or more of the following characteristics, an ability to improving gastrointestinal and urogenital health comprising detecting an ability to: adhere to gastrointestinal, vaginal and uroepithelial cells by electrostatic, hydrophobic or specific adhesins including a collagen binding protein; pass through the stomach and reach the small and large intestine; grow and persist in the gastrointestinal and urogenital tracts; inhibit the adhesion of gastrointestinal an urogenital pathogens including organisms which cause urinary tract infection, bacterial vaginosis and yeast vaginitis; coaggregate to form a balanced flora; produce acid and other substances such as hydrogen peroxide and/or bacteriocins and bacteriocin-like compounds which inhibit pathogen growth; produce biosurfactant or related by-products of growth which interfere with adhesion of pathogens to cells and materials; resist antimicrobial agents, such as nonoxynol-9 spermicide; and/or enhance the host's immune function to further maintain a healthy urogenital flora. A preferred probiotic organism is one or more species of Lactobacillus and extracts or by-products thereof such as proteins or peptides or amino acids.

The preferred strains of lactobacilli within the scope of this invention are aerobic, microaerophilic and anaerobic isolates. A most preferred Lactobacillus species is L. fermentum RC-14. Another preferred Lactobacillus species is L. rhamnosus GR-1. Still another preferred lactobacillus species is L. fermentum B-54.

The preferred strains of lactobacilli within the scope of this invention are anaerobic and microaerophilic isolates.

By “prebiotic” is meant a nonmetabolized, nonabsorbed substrate that is useful for the host which selectively enhances the growth and/or the metabolic activity of a bacterium or a group of bacteria. A prebiotic also includes a nutrient utilized by lactobacilli or bifidobacteria to stimulate and/or enhance growth of lactobacilli or bifidobacteria relative to pathogenic bacteria.

Also defined within the present invention are compositions suitable for establishing, maintaining or restoring a healthy gastrointestinal and urogenital flora in females throughout life which comprise one or more Lactobacillus viable whole cells, non-viable whole cells or cell wall fragments and a pharmaceutically acceptable carrier. By “throughout life” is meant in the neonatal period, during childhood and in the pre-menopausal and post-menopausal periods. By “healthy gastrointestinal and urogenital flora” is meant flora that is predominantly colonized by non-pathogenic organisms and where there are no signs or symptoms of infection or disease.

In a preferred aspect, the Lactobacillus is aerobically, microaerophilically or anaerobically grown and may be selected from the group consisting of Lactobacillus casei, L. acidophilus, L. plantarum, L. fermentum, L. brevis, L jensenii, L. crispatus, L. rhamnosus, L. reuteri, L. paracasei, L. gasseri, L. cellobiosis, L. delbrueckii, L. helveticus, L. salivarius, L. collinoides, L. buchneri, L. rogosae and L. bifidium.

The Lactobacillus may be microaerophilically or anaerobically grown and selected from the group consisting of Lactobacillus rhamnosus (GR-1 (ATCC 55826), L. rhamnosus GR-2 (ATCC 55915), L. rhamnosus GR-3 (ATCC 55917), L. rhamnosus GR-4 (ATCC 55916), L. rhamnosus RC-9, L. rhamnosus RC-17 (ATCC 55825), L. casei var alactosus RC-21, L. casei NRC 430, L. casei ATCC 7469, L. rhamnosus 81, L. rhamnosus 76, L. rhamnosus 36W, L. rhamnosus 36g, L. casei RC-65, L. casei RC-15, L. casei 558, L. casei, RC-21, L. casei 55, L. casei 8, L. casei 43, L. plantarum RC-12 (ATCC 55895), L. acidophilus RC-25, L. plantarum RC-19, L. jensenii RC-11 (ATCC 55901), L. acidophilus ATCC 4357, L. acidophilus 2099 B, L. acidophilus 2155C, L. acidophilus T-13, L. acidophilus 1807B, L. acidophilus RC-16, L. acidophilus RC-26, L. acidophilus RC-10, L. acidophilus RC-24, L. acidophilus RC-13, L. acidophilus RC-14, L. acidophilus RC-12, L. acidophilus RC-22, L. acidophilus 2099B, L. acidophilus 2155C, L. acidophilus T-13, L. plantarum ATCC 8014, L. plantarum UH 2153, L. plantarum 260, L. plantarum RC-20, L. plantarum 75, L. plantarum RC-6, L. fermentum A-60, L. fermentum B-54 (identical ribotype to RC-14) (ATCC 55920), L. cellobiosis RC-2, L. crispatus 1350B and L. crispatus 2142B.

In a further embodiment, the present invention describes a method of administering probiotic organisms orally for restoring a healthy urogenital and intestinal flora over the various life cycle stages of women including pregnancy and post-menopause, wherein the flora is dominated by Mobiluncus, Gardnerella, Bacteroides, Fusobacterium, Prevotella, Peptostreptococcus, Porphyromonas, Mycoplasma or group B streptococci, or Escherichia coli, Staphylococcus sp., Enterococcus sp, Klebsiella sp, Pseudomonas sp, Streptococcus sp, Proteus sp, and other Gram negative (such as coliforms) and Gram positive pathogens which cause urinary tract infections and gastrointestinal infections, and yeast including Candida albicans, for example.

In a preferred embodiment, the Lactobacillus species will produce biosurfactants active against urogenital pathogens including those that cause urinary tract infections, bacterial vaginosis and yeast vaginitis such as Mobiluncus, Gardnerella, Bacteroides, Fusobacterium, Prevotella, Peptostreptococcus, Porphyromonas, Mycoplasma or group B streptococci, or Escherichia coli, Enterococcus sp, Klebsiella sp, Pseudomonas sp, Streptococcus sp, Proteus sp, and yeast.

In another embodiment, the Lactobacillus species will inhibit growth and adhesion of enteric pathogens to gastrointestinal surfaces including those that cause enteric infections. Such inhibition of enteric pathogens is at least partly due to the production of biosurfactants active against such pathogens including, salmonella, shigella, listeria, campylobacter and clostridium, for example.

Biosurfactants produced by lactobacilli significantly inhibit the binding of urogenital and gastrointestinal pathogens to surfaces. These biosurfactants contain carbohydrate and proteinaceous compounds. Biochemical analysis using PAGE, affinity chromatography, and amino acid sequencing of biosurfactant produced by L. fermentum RC-14 evidences a 26 kD protein which binds to collagen. This protein, and others which also bind to collagen, play an important role in the colonization by lactobacilli of the vaginal vault. This 26 kD protein is also understood, in accordance with the present invention to play an important role in the protection of the heart against urogenital pathogens.

Separation and detection of biosurfactants produced by lactobacilli may be preferably accomplished by the SELDI technique (Surface Enhanced Laser desorption/ionization). By “SELDI system” is meant a method which uses protein chips which contain chemically or biologically treated surfaces that specifically interact with or bind the proteins of interest. The protein chips are inserted into a reader which provides an accurate mass profile of the proteins bound to each chip in just a few minutes.

In a further embodiment the present invention provides a method for selecting lactobacilli and bifidobacteria useful for improving gastrointestinal and urogenital health. Criteria are provided herein for characterizing a selected Lactobacillus or Bifidobacterium as candidates for the contemplated methods and compositions of the present invention. The probiotic organisms will exhibit some or all of the following criteria: an ability to: adhere to vaginal and uroepithelial cells by electrostatic, hydrophobic or specific adhesins including but not limited to a collagen binding protein; pass through the stomach and reach the small and large intestine and urogenital tract; grow and persist in the gastrointestinal and urogenital tracts; inhibit the adhesion of urogenital pathogens including organisms which cause urinary tract infection, bacterial vaginosis and/or yeast vaginitis; coaggregate to form a balanced flora; produce acid and other substances such as hydrogen peroxide and/or bacteriocins and bacteriocin-like compounds which inhibit pathogen growth; produce biosurfactant or related by-products of growth which interfere with adhesion of pathogens to cells and materials; resist antimicrobial agents, such as nonoxynol-9 spermicide; and/or enhance the host's immune function to further maintain a healthy urogenital flora.

Although this invention is not intended to be limited to any particular mode of application, oral administration. of the compositions are preferred. One probiotic organism may be administered alone or in conjunction with a second, different probiotic organism. By “in conjunction with” is meant together, substantially simultaneously or sequentially. The compositions may be administered in the form of tablet, pill or capsule, for example. One preferred form of application involves the preparation of a freeze-dried capsule comprising the composition of the present invention. It has been found that a capsule comprising about 10⁹ probiotic organisms is suitable. In accordance with the present invention a capsule may contain one single or two or more different species of probiotic organism(s).

By “therapeutically effective amount” as used herein is meant an amount of probiotic organism, e.g., lactobacillus, high enough to significantly positively modify the condition to be treated but low enough to avoid serious side effects (at a reasonable benefit/risk ratio), within the scope of sound medical judgment. A therapeutically effective amount of lactobacillus will vary with the particular goal to be achieved, the age and physical condition of the patient being treated, the severity of the underlying disease, the duration of treatment, the nature of concurrent therapy and the specific lactobacillus employed. For example, a therapeutically effective amount of probiotic organism administered to a child or a neonate will be reduced proportionately in accordance with sound medical judgment. The effective amount of lactobacillus will thus be the minimum amount which will provide the desired attachment to epithelial cells. For example, the presence of 5×10⁹ bacteria, as viable or non-viable whole cells, in 0.05 ml solution of phosphate buffered saline solution, or in 0.05 ml of suspension of agar, or the dry weight equivalent of cell wall fragments, is effective when administered in quantities of from about 0.05 ml to about 20 ml.

A decided practical advantage is that the probiotic organism, e.g. Lactobacillus, may be administered in a convenient manner such as by the oral, intravenous (where non-viable), or suppository (vaginal or rectal) routes. Depending on the route of administration, the active ingredients which comprise probiotic organisms may be required to be coated in a material to protect said organisms from the action of enzymes, acids and other natural conditions which may inactivate said organisms. In order to administer probiotic organisms by other than parenteral administration, they should be coated by, or administered with, a material to prevent inactivation. For example, probiotic organisms may be co-administered with enzyme inhibitors or in liposomes. Enzyme inhibitors include pancreatic trypsin inhibitor, diisopropylfluorophosphate (DFP) and trasylol. Liposomes include water-in-oil-in-water P40 emulsions as well as conventional and specifically designed liposomes which transport lactobacilli or their by-products to the urogenital surface.

The probiotic organisms may also be administered parenterally or intraperitoneally. Dispersions can also be prepared, for example, in glycerol, liquid polyethylene glycols, and mixtures thereof, and in oils.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage. The carrier can be a solvent or dispersion medium containing, for example, water, ethanol, polyol (for example, glycerol, propylene glycol, liquid polyethylene glycol, and the like), suitable mixtures thereof and vegetable oils. The proper fluidity can be maintained, for example, by the use of a coating such as lecithin, by the maintenance of the required particle size in the case of dispersion. In many cases it will be preferable to include isotonic agents, for example, sugars or sodium chloride. Prolonged absorption of the injectable compositions can be brought about by the use in the compositions of agents delaying absorption, for example, aluminum monostearate and gelatin..

Sterile injectable solutions are prepared by incorporating the probiotic organisms in the required amount in the appropriate solvent with various of the other ingredients enumerated above, as required, followed by filtered sterilization. Generally, dispersions are prepared by incorporating the various sterilized probiotic organisms into a sterile vehicle which contains the basic dispersion medium and the required other ingredients from those enumerated above. In the case of sterile powders for the preparation of sterile injectable solutions, the preferred methods of preparation are vacuum-drying and the freeze-drying technique which yield a powder of the active ingredient plus any additional desired ingredient from previously sterile-filtered solution thereof.

When the probiotic organisms are suitably protected as described above, the active compound may be orally administered, for example, with an inert diluent or with an assimilable edible carrier, or it may be enclosed in hard or soft shell gelatin capsule, or it may be compressed into tablets designed to pass through the stomach (i.e., enteric coated), or it may be incorporated directly with the food of the diet. For oral therapeutic administration, the probiotic organisms may be incorporated with excipients and used in the form of ingestible tablets, buccal tablets, troches, capsules, elixirs, suspensions, syrups, wafers, and the like. Compositions or preparations according to the present invention are prepared so that an oral dosage unit form contains about 1×10⁹ viable or non-viable e.g., lactobacilli per ml.

The tablets, troches, pills, capsules, and the like, as described above, may also contain the following: a binder such as gum tragacanth, acacia, corn starch or gelatin; excipients such as dicalcium phosphate; a disintegrating agent such as corn starch, potato starch, alginic acid, and the like; a lubricant such as magnesium stearate; and a sweetening agent such as sucrose, lactose or saccharin may be added or a flavoring agent such as peppermint, oil or wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, pills or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the probiotic organism may be incorporated into sustained-release preparations and formulations.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of the probiotic organisms calculated to produce the desired therapeutic effect in association with the required pharmaceutical carrier. The specification for the novel dosage unit forms of the invention are dictated by and directly depending on (a) the unique characteristics of the probiotic organism and the particular therapeutic effect to be achieved, and (b) the limitations inherent in the art of compounding such probiotic for the establishment and maintenance of a healthy urogenital flora.

The probiotic organism is compounded for convenient and effective administration in effective amounts with a suitable pharmaceutically or food acceptable carrier in dosage unit form as hereinbefore disclosed. A unit dosage form can, for example, contain the principal active compound in an amount approximating 10⁹ viable or non-viable, e.g., lactobacilli, per ml. In the case of compositions containing supplementary ingredients such as prebiotics, the dosages are determined by reference to the usual dose and manner of administration of the said ingredients.

By “pharmaceutically-acceptable carrier” as used herein is meant one or more compatible solid or liquid filler diluents, encapsulating substances or foods or drinks, such as yogurt, for example. By “compatible” as used herein is meant that the components of the composition are capable of being comingled without interacting in a manner which would substantially decrease the pharmaceutical efficacy of the total composition under ordinary use situations. The pharmaceutical carrier in accordance with the present invention also is also contemplated to encompass microbial nutrients including specific prebiotics which differentially stimulate the healthy flora, and factors such as antimicrobial compounds, naturally occurring peptides, herbs, vitamins, minerals and plant material, which are active against urogenital pathogens.

Some examples of substances which can serve as pharmaceutical carriers are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tabletting agents, stabilizers, anti-oxidants and preservatives, can also be present.

Accordingly, in a preferred form of establishing, maintaining or restoring a healthy gastrointestinal and urogenital flora, the patient is orally administered a therapeutically effective amount of at least one probiotic organism and a pharmaceutically acceptable carrier in accordance with the present invention. A most preferred probiotic organism is a Lactobacillus. Preferably, the Lactobacillus is selected from the group comprising L. rhamnosus, L. casei ss alactosus, L. fermentum and L. brevis. Most preferably, the lactobacillus is either L. rhamnosus GR-1 or L, fermentum B-54 or L. acidophilus RC-14.

Another preferred composition comprises at least one probiotic organism and a prebiotic and a pharmaceutically acceptable carrier. A preferred prebiotic is insulin.

The introduction or administration of probiotics to pregnant women in accordance with the present invention will provide protection against infections such as bacterial vaginosis, Group B streptococci, urinary tract infections and others which are capable of adversely affecting the fetus, the newborn and the mother. Accordingly, in a preferred method of establishing a healthy, normal urogenital flora in women before or during pregnancy, a vaginal culture is obtained from the individual and the culture is assayed for the presence of the lactobacilli or bifidobacteria. Selected lactobacilli or bifidobacteria are isolated, purified, grown and optionally frozen and stored (e.g., commercially) for future use by the donor. Alternatively, selected lactobacilli or bifidobacteria are orally or vaginally re-administered in a therapeutically effective amount and form to the donor. In a preferred embodiment at least one probiotic organism is isolated from a donor in need of flora restoration or maintenance. Isolated organisms are resuspended in a pharmaceutical carrier and grown to a concentration permitting the reintroduction or reimplantation of about 10⁹ organisms/ml. Reimplanted probiotic organisms are preferably administered about twice a week for about one week to about 52 weeks and most preferably for about one week to about 36 weeks. Orally reintroduced probiotic organisms are preferably administered daily for about one week to about 52 weeks and most preferably for about one week to about 40 weeks.

The introduction or administration of lactobacilli probiotics to the intestine and passage onto the urogenital tract, and their subsequent production of anti-pathogenic products (e.g., biosurfactants, acids, hydrogen peroxide, bacteriocins) stimulates the immune response against infection and disease and reduces the risk of medical device associated infections. While not wishing to be bound by a particular mechanism, host responses are stimulated which inhibit pathogens and/or create a microenvironment less conducive to pathogen spread in women. Accordingly, in a preferred embodiment of stimulating host responses, a medical device is contacted or coated with lactobacillus at a concentration of about 10⁹ organisms/ml prior to introduction into a patient in need of such device. Medical devices contemplated by the present invention include but are not limited to: intrauterine devices, catheters, stents, drainage lines, intravenous lines, diaphragms, implants, screws, sutures, pads and tampons, for example.

Although the present invention is not bound by any one theory or mode of operation, it is believed that, at least to some degree, a combination of coaggregation of Lactobacillus and the production by Lactobacillus of one or more inhibitory substances may be responsible for excluding pathogens and/or reducing their numbers at the site of a gastrointestinal or genito-urinary infection.

From the standpoint of physical exclusion, the attachment of Lactobacillus acts as a block to pathogens by inhibiting access to receptor sites. Although complete exclusion of pathogens theoretically can occur, the most common finding of the results of the present invention is that there is a reduction in pathogen numbers compared to probiotic organisms, e.g., lactobacilli. In other words, although some probiotic organisms may not completely exclude pathogens, they are still capable of interfering with pathogen colonization in vivo.

### EXAMPLE 1

The introduction or administration of Lactobacilli restored the urogenital healthy flora and replaced the abnormal biofilm with a healthy biofilm in women having signs of preterm labor.

Vaginal swabs were collected and analyzed by a Nugent scoring system which grades the flora as normal (score of 0 and dominated by lactobacilli) to 10 (absence of lactobacilli and flora dominated by Gram negative anaerobic bacterial vaginosis pathogens).

Of 39 women presenting with signs of preterm labor, 23 were diagnosed as having abnormal lactobacilli presence (Nugent scores 4-6) and 16 actually had bacterial vaginosis (B.V.) (Nugent scores >7).

Oral administration of lactobacilli to restore the lactobacilli flora provided an intervention that lowered the risk of pregnant women going on to deliver their baby preterm. (Table 1)

TABLE 1
PRETERM LABOR STUDY

| | | G.A. | | | | | | Group | |
|---|---|---|---|---|---|---|---|---|---|
| Sample | | @ | Vaginal | | | Clue | Nugent | B | |
| # | G.A. | Del. | Discharge | pH | Whiff | cells | Score | Step. | Comment |
| 4001 | 34 + 1 | | No | N/A | −ve | −ve | 2 | −ve | |
| 4002 | 33 + 6 | | White | 4.5 | −ve | −ve | 8 | −ve | B.V. |
| 4003 | 32 + 6 | | White | 5.0 | −ve | −ve | 6 | −ve | |
| 4004 | 34 | | White | 5.0 | −ve | −ve | 6 | −ve | |
| 4005 | 32 + 6 | | No | 5.0 | −ve | −ve | 8 | −ve | B.V. |
| 4006 | 34 + 5 | | White | 6.0 | −ve | −ve | 8 | −ve | B.V. |
| 4007 | 32 + 1 | | No | 4.0 | +ve | −ve | 8 | −ve | B.V. |
| 4008 | — | | — | — | — | — | — | — | — |
| 4009 | 36 + 4 | 36 + 4 | White | 5.0 | −ve | −ve | 7 | −ve | B.V. |
| 4010 | 33 + 6 | | White | 4.7 | −ve | −ve | 8 | −ve | B.V. |
| 4011 | 33 | | Gray | 4.7 | −ve | −ve | 6 | −ve | |
| 4012 | 33 + 2 | | White | 4.0 | −ve | −ve | 6 | −ve | |
| 4013 | 33 | | No | 5.2 | −ve | −ve | 8 | −ve | |
| 4014 | 32 + 4 | 36 | No | 5.0 | −ve | −ve | 8 | −ve | B.V. |
| 4015 | 30 + 3 | 30 + 4 | No | 5.0 | −ve | −ve | 8 | −ve | B.V. |
| 4016 | 29 + 1 | | White | 4.7 | +ve | −ve | 7 | −ve | B.V. |
| 4017 | 32 | | White | 4.7 | −ve | −ve | 8 | −ve | B.V. |
| 4018 | 30 | | No | 4.7 | −ve | −ve | 6 | −ve | ? Strep. |
| 4019 | 33 | 36 + 1 | White | 4.7 | −ve | −ve | 1 | −ve | Lacto. |
| | | | | | | | | | acidophilus |
| 4020 | 34 | | White | 4.7 | −ve | −ve | 6 | −ve | Lactis Lactis |
| 4021 | 35 +3 | | White | 4.5 | −ve | −ve | 8 | −ve | B.V. |
| 4022 | 33 + 3 | | Yellow | N/A | −ve | −ve | 4 | −ve | Lacto. |
| | | | | | | | | | rhamnosus |
| 4023 | — | | — | — | — | — | — | — | — |
| 4024 | 24 | | White | 5.0 | −ve | −ve | 7 | −ve | B.V. |
| 4025 | 32 | 35 + 2 | No | 5.3 | −ve | −ve | 4 | −ve | Lacto |
| | | | | | | | | | acidophilus |
| 4026 | 34 + 3 | | White | 5.0 | −ve | +ve | 8 | −ve | B.V. |
| 4027 | 32 | | White | 4.7 | −ve | −ve | 3 | N/A | L.c. |
| | | | | | | | | | raffinolactis |
| 4028 | N/A | | No | 5.8 | −ve | +ve | 3 | −ve | Lactis Lactis |
| 4029 | 32 + 2 | | White | 4.4 | −ve | −ve | 2 | N/A | Lacto. delb. |
| | | | | | | | | | delb. |
| 4030 | 24 + 5 | | Gray- | 4.4 | −ve | −ve | 2 | N/A | Lacto. delb. |
| | | | | | | | | | delb. |
| 4031 | 23 + 3 | 26 | White | 5.0 | N/A | −ve | 3 | N/A | |
| 4032 | 36 + 2 | 36 + 2 | No | 4.4 | −ve | −ve | 1 | −ve | Lacto. delb. |
| | | | | | | | | | delb. |
| 4033 | 01/32 | | No | 4.4 | −ve | −ve | 3 | N/A | Lacto. |
| | | | | | | | | | crispatus |
| 4034 | 32 + 3 | 32 + 5 | White | 4.2 | −ve | −ve | 0 | −ve | |
| 4035 | 30 + 2 | | No | 4.4 | −ve | −ve | 6 | N/A | |
| 4036 | — | — | — | — | — | — | — | — | |
| 4037 | 24 | | No | 4.7 | −ve | −ve | 5 | −ve | Lacto. |
| | | | | | | | | | crispatus |
| 4038 | 24 + 5 | | White | 5.3 | −ve | −ve | 8 | −ve | B.V. |
| 4039 | 36 + 1 | | No | 7.0 | −ve | −ve | 7 | N/A | B.V. |
| 4040 | 30 | | White | 5.0 | −ve | −ve | 7 | +ve | B.V. |
| 4041 | 29 | | White | 4.7 | −ve | −ve | 2 | | |

| Bacterial Morphotype | None | 1+ | 2+ | 3+ | 4+ |
|---|---|---|---|---|---|
| Large gram- | 4 | 3 | 2 | 1 | 0 |
| positive rod | | | | | |
| Small gram- | 0 | 1 | 2 | 3 | 4 |
| negative/variable | | | | | |
| rod | | | | | |
| Curved | 0 | 1 | 1 | 2 | 2 |
| negative/variable | | | | | |
| rod | | | | | |

Score of 0-3 points, Normal; 4-6, intermediate; 7-10 B.V. G.A. = gestational age; N/A = not available; ve = vaginal epithelial cells

Score of 0-3 points, Normal; 4-6, intermediate; 7-10 B.V. G.A.=gestational age; N/A=not available; ve=vaginal epithelial cells

### EXAMPLE 2

SELDI (surface enhanced laser desorption/ionization) was used to separate, detect and analyze native proteins at the femtomole level without using labeling or time consuming biochemical analytical systems. The SELDI system was used to quickly and accurately determine whether clinically important strains of lactobacilli expressed collagen binding proteins.

Four Lactobacillus strains were tested. L. fermentum RC-14 was selected because of its potent biosurfactant inhibitory activity against many urogenital pathogens. L. rhamnosus GR-1 and 36 also produce biosurfactant, and are also inhibitory to enterococci.

The organisms were grown in MRS broth overnight, harvested and the biosurfactant isolated by incubating the organisms for two hours at room temperature.

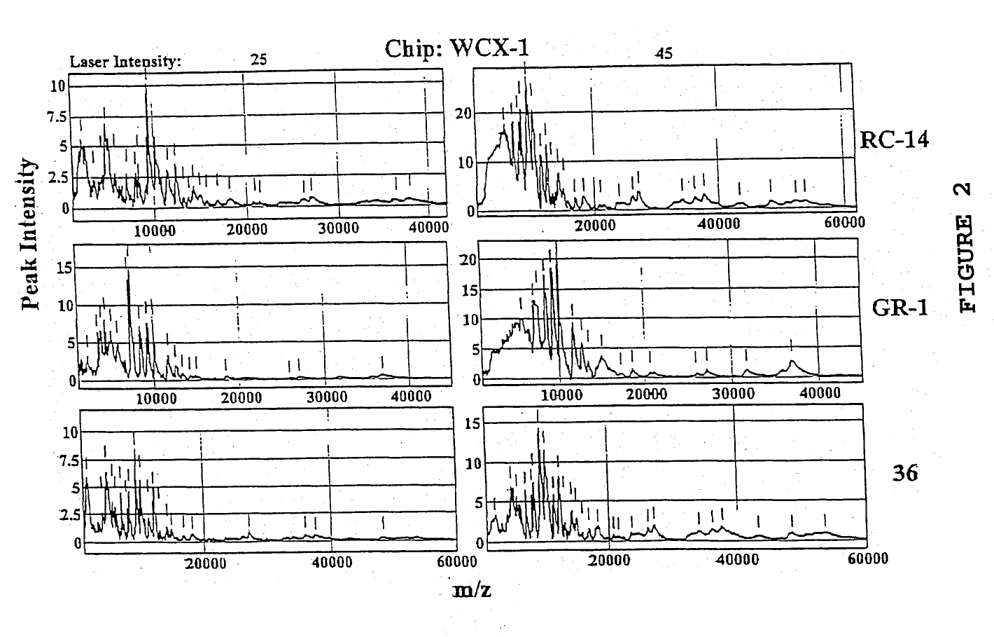

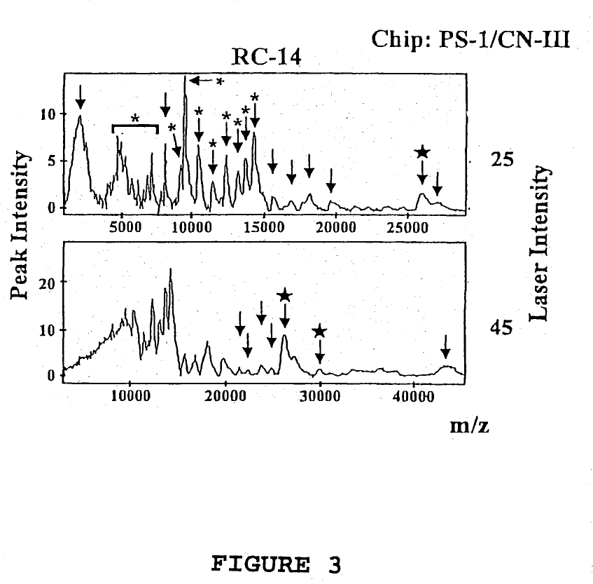

SELDI System. The resultant data showed the presence of several collagen binding proteins in the RC-14 biosurfactant preparation tested with calf skin and human placental collagen, particularly at 1.9, 4.7, 9.4, 14.2, 26 and 37 kDa (FIGS. 2 and 3). Strains GR-1, RC-14 and 36 contained both a 26 kD and 36 kD protein. Further analysis-of the biosurfactants showed the presence of sixteen amino acids present in varying amounts. (Table 2)

TABLE 2
AMINO ACID COMPOSITION OF HYDROLYZED LACTOBACILLUS BIOSURFACTANTS

| Bio- | **AMINO ACID COMPOSITION (MOLE %) | | | | | | | | | | | | | | | |
|---|---|---|---|---|---|---|---|---|---|---|---|---|---|---|---|---|
| surfactant | Asx* | Thr | Ser | Glx* | Gly | Ala | Val | Met | Ile | Leu | Phe | Tyr | His | Lys | Arg | Pro |
| 36*** | 8.23 | 3.6 | 2.98 | 10.59 | 8.4 | 33.98*** | 5.41 | 1.07 | 3.58 | 5.82 | 1.29 | 1.97 | 1.27 | 5.9 | 2.39 | 3.52 |
| GR-1 | 10.3 | 7.0 | 12.3 | 18.4 | 18.7 | 7.86 | — | — | 2.02 | 10.7 | — | — | 4.1 | — | 1.05 | 6.94 |
| RC-14 | 10.4 | 4.67 | 5.81 | 12.5 | 10.1 | 8.91 | 6.19 | 1.02 | 3.24 | 9.5 | 2.67 | 3.54 | 3.64 | 7.6 | 5.76 | 4.52 |

* Sample preparation resulted in the deamination of asparagine and glutamine into aspartic acid and glutamic acid, respectively.
** Due to analysis conditions cysteine and tryptophan could not be accurately quantified.
*** The unlikely high values indicated the presence of free alanine in the sample.

### EXAMPLE 3

When the patient in need of flora restoration or maintenance is healthy, urogenital organisms are recovered, cultured and the main healthy species isolated and stored. If and when the person has a depleted urogenital flora at some later point in her life, such as during pregnancy or during a urogenital infection, the originally isolated organisms are cultured, and re-implanted vaginally or re-administered orally. This approach will both personalize the therapy and utilize organisms which are known to have been associated with the person's health and recognized as “self”-organisms by their urogenital system at one stage in life.

### EXAMPLE 4

L. fermentum RC-14 has been shown to express a biosurfactant substance capable of inhibiting the adhesion to polystyrene plates of Gardnerella vaginalis by 84%, Bacteroides fragilis ATCC 25285 by 95%, and Group B streptococcus by 100%, and uropathogenic Enterococcus faecalis by up to 90%. Given this data, it is apparent that the lactobacilli inhibit organisms responsible for bacterial vaginosis and vaginitis, including Mobiluncus, Fusobacterium, Prevotella, Peptostreptococcus, Porphyromonas and Mycoplasma species.

Such inhibition of colonization of surfaces of these pathogens is clinically relevant and significant. The only way to eradicate these organisms is the use of antibiotics, and this can have significant side effects particularly for the pregnant mother and fetus. Antimicrobial therapy not only affects the pathogenic organisms, but also impacts the extant vaginal lactobacilli and bifidobacteria, by depleting such healthy flora. In addition, pathogenic bacteria exist in biofilms, and are able to resist antibiotic treatment and thereby further increasing the problems of infection. Thus, even after antibiotic therapy to treat bacterial vaginosis, the pathogens still exist for duration of pregnancy, thereby jeopardizing the health of the mother and fetus.

The contemplated method of administering the probiotics includes daily intake for the first 12 months to 4 years of life, at a time when the male and female newborn is particularly susceptible to urinary infections, which often lead to kidney infection; renal impairment and renal failure. The method of administering the probiotics include daily from puberty to menopause to establish a prolonged healthy urogenital flora during reproductive years, and then altering the composition of the probiotic for daily use post-menopause.

### EXAMPLE 5

Naturally occurring substances, such as vitamins, minerals, plants and human peptides have been shown to have activity against uropathogenic organisms. For example, cecropin P1 and Fall-39, vitamin C, cranberry extracts, and other herbs. The critical aspect is the ability of the substances to selectively affect uropathogens as distinct from pathogenic organisms.

FALL-39 and Cecropin P1 are examples of natural peptides, (Agerberth et al. 1991; Lee et al. 1989) which, according to the mass-spectroscopy the purified FALL-39 fraction had a molecular mass of 4715.23 and the purified cecropin P1 gave a molecular mass of 3338.3, both of which are active against urogenital pathogens (for example Escherichia coli HU734, Enterococcus faecalis 1131, Pseudomonas aeruginosa AK1, Proteus mirabilis 28cii, Klebsiella pneumoniae 3a, and Staphylococcus epidermidis 1938) but not so much against Lactobacillus strains (for example L. rhamnosus GR-1, L. rhamnosus 81, L. fermentum RC-14, L. fermentum B-54, and L. plantarum RC-20).

Action of the peptide was considered bactericidal if less than 0.1% of original number of bacteria remained viable following the treatment. Resistant organisms were also plated from the 100 μmol 1⁻¹ well to confirm presence of any inhibition. Both peptides were found to be active against Gram-negative urogenital pathogens. The mode of action of FALL-39 was found to be bactericidal for E. coli and bacteriostatic for both K. pneumoniae, and P. aeruginosa, whereas Cecropin P1 acted bacteriocidally toward to all three sensitive uropathogens. In addition, we demonstrated that these peptides are active against clinically important strains of Gram-negative urogenital pathogens including previously untested organism K. pneumoniae

When lactobacilli were used as indicator organisms, all strains were highly resistant to Cecropin P1 (MICs>100 μM). In a case of FALL-39, four strains, L. rhamnosus GR-1, L. rhamnosus 81, L. plantarum RC-20 and L. fermentum RC-14 were resistant, and one strain, L. fermentum B-54 was susceptible only at 100 μM. No statistically significant differences were found between viable counts of the resistant strains of lactobacilli in the well with highest concentration of peptides (100 μmol 1⁻¹) and in control wells. FALL-39 appears to act bacteriostatically against L. fermentum B-54 (Table 3).

TABLE 3
Minimal inhibitory concentrations (MIC) of FALL-39 and
cecropin P1 against uropathogenic microorganisms.

| | MIC (μmol 1⁻¹ | MIC (μmol 1⁻¹ M) |
|---|---|---|
| Organism | M) FALL-39 | Cecropin P1 |
| E. coli | 25 | 1.56 |
| Ps. aeruginosa | 12.5 | 25 |
| Kl. pneumoniae | 50 | 1.56 |
| Pr. mirablis | >100 | >100 |
| Ent. faecalis | >100 | >100 |
| Staph. epidermidis | >100 | >100 |

What is claimed is:

1. A method of reducing the risk of preterm labor comprising orally administering to a mammalian subject a therapeutically effective amount of at least one probiotic organism and a pharmaceutically acceptable carrier.

2. The method of claim 1 further comprising the administration of a therapeutically effective amount of at least one second probiotic organism.

3. The method of claim 1 wherein said probiotic organism is a Lactobacillus.

4. The method of claim 2 wherein said second probiotic organism is a Bifidobacterium.

5. The method of claims 1 or 2 further comprising the administration of a therapeutically effective amount of a prebiotic.

6. The method of claim 1 wherein said probiotic organism is selected from the group consisting of L. rhamnosus, L. acidophilus, L. fermentum, L. casei, L reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosae, or L. bifidum.

7. The method of claim 2 wherein said second probiotic organism is selected from the group consisting of B. bifidum, B. breve, B. adolescentis, or B. longum.

8. An ex vivo method of establishing a healthy gastrointestinal and urogenital flora in a females comprising orally administering to a mammalian subject at least one probiotic organism isolated from said female and a pharmaceutically acceptable carrier.

9. The method of claim 8 wherein said probiotic organism is isolated from the genito-urinary tract of said female.

10. An ex vivo method of restoring healthy gastrointestinal and urogenital flora in females in need thereof comprising orally administering to a mammalian subject at least one probiotic organism isolated from the individual and a pharmaceutically acceptable carrier.

11. A method of maintaining a healthy gastrointestinal tract in females comprising orally administering to a mammalian subject a therapeutically effective amount of at least one probiotic organism and a pharmaceutically acceptable carrier.

12. The method of claim 11 further comprising the administration of a therapeutically effective amount of at least one second probiotic organism.

13. The method of claim 11 wherein said probiotic organism is a Lactobacillus.

14. The method of claim 12 wherein said second probiotic organism is a Bifidobacterium.

15. The method of claim 11 or 12 further comprising the administration of a therapeutically effective amount of a prebiotic.

16. A pharmaceutical composition comprising at least one probiotic organism and a pharmaceutically acceptable carrier.

17. A method for reducing the risk of bacterial vaginosis and bacterial vaginosis pathogens comprising orally administering to a mammalian subject to a mammalian subject a therapeutically effective amount of at least one probiotic organism and a pharmaceutically acceptable carrier.

18. The method of claim 17 further comprising the administration of a therapeutically effective amount of at least one second probiotic organism.

19. The method of claim 17 wherein said probiotic organism is a Lactobacillus.

20. The method of claim 18 wherein said second probiotic organism is a Bifidobacterium.

21. The method of claims 17 or 18 further comprising the administration of a therapeutically effective amount of a prebiotic.

22. The method of claim 17 wherein said probiotic organism is selected from the group consisting of L. rhamnosus, L. acidophilus, L. fermentum, L. casei, L reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosae, or L. bifidum.

23. The method of claim 18 wherein said second probiotic organism is selected from the group consisting of B. bifidum, B. breve, B. adolescentis, or B. longum.

24. A method of stimulating host responses to pathogens comprising contacting a medical device with a therapeutically effective amount of at least one probiotic organism and a pharmaceutically acceptable carrier prior to introduction into a patient in need of such device. a therapeutically effective amount of at least one probiotic organism and a pharmaceutically acceptable carrier.
